(19) United States
(12) Patent Application Publication
(10) Pub. No.: US 2023/0181694 A1
(43) Pub. Date: Jun. 15, 2023

(54) TREATMENT OF RESPIRATORY VIRAL INFECTIONS

(71) Applicant: KYON BIOTECH AG, Zürich (CH)

(72) Inventor: Slobodab TEPIC, Zürich (CH)

(73) Assignee: KYON BIOTECH AG, Zürich (CH)

(21) Appl. No.: 17/924,905

(22) PCT Filed: May 11, 2021
(86) PCT No.: PCT/EP2021/062513

§ 371 Date: Nov. 11, 2022

(30) Foreign Application Priority Data
May 14, 2020 (EP) 20174730.0

(51) Int. Cl. A61K 38/28; A61K 38/50

(52) U.S. Cl. CPC A61K 38/28; A61K 38/50

(57) ABSTRACT

The present invention relates to the treatment of respiratory viral infections, including those caused by coronaviruses.

The present invention relates to the treatment of respiratory viral infections, including those caused by Coronaviruses.

It is spring of 2020 and the world is in the midst of Covid-19 infection with more than 3 million confirmed cases and 200′000 deaths.

There is a large number of potential drugs in testing, those developed in the past for other infections and those specifically targeting SARS-CoV-2, the coronavirus that causes Covid-19 disease. It is now about four months since an infectious lung disease was recognized as a novel one in the Chinese city of Wuhan. The report from China to the World Health Organization on Dec. 31, 2019, was followed on Jan. 11, 2020 by the publication of the molecular structure of the SARS-CoV-2 virus, initiating a worldwide, massive effort to find both, a treatment and a vaccination.

Until this time, no promising treatment has emerged, several have already been written off after disappointing trial outcomes. With billions of people around the world under lockdown, measures aimed at slowing the spread of the infection, expectations and hopes of the general public and most of the medical establishment are that the percentage of populations needed to reach herd immunity will get vaccinated in the next few years. Meanwhile, millions more will die and many more will suffer from the disease and economic consequences that this pandemic has brought onto the world economy.

The primary focus of research towards treatment is on the molecular details and biological functions of some of the 29 identified SARS-CoV-2 proteins. The main strategy is to disrupt the process of viral replication at any of the identified steps from the virus entry into cells, replication, and shedding of the newly formed viruses.

Meanwhile, a number of clinical trials have been and are being conducted based on existing drugs in hope of finding quicker help for those patients that get seriously ill or die.

According to the present invention, a novel strategy is provided, which is based on the rapid killing of infected cells before new viral particles are produced and shed. The selectivity of the attack on the infected cells is not based on the molecular structure of the virus, but on fundamental changes in cellular metabolic processes of infected cells. The main characteristic of any viral infection is the recruitment of the cellular synthetic pathways towards replication of viral proteins and nucleic acids leading to enhanced exchanges with the environment, including an increased requirement for amino acids. While producing viral proteins the cell still needs to support its own needs for protein maintenance. In that metabolic state, depleting one or more amino acids can lead to a rapid demise of the cell and thus preventing it from spreading the infection. In contrast thereto, healthy cells are equipped with mechanisms of controlling mass transport across the membrane and of internal recycling of amino acids from protein turnover, efficient enough to enable them to survive for days or even weeks of extracellular amino depletion.

Amino acid depletion has been clinically used for decades in oncology (Garcia-Bermudez J, Williams R T, Guarecuco R, Birsoy K. Targeting extracellular nutrient dependencies of cancer cells. Mol Metab. 2020; 33: 67-82). Most successful so far has been the use of asparaginase in treating blood cancers, particularly in childhood Acute Lymphoblastic Leukemia. Further, depletion of arginine has also attracted much interest and there are numerous clinical trials with arginine degrading enzymes such as arginase and arginine deiminase.

A first aspect of the present invention is a medicament comprising at least one amino acid degrading enzyme for use in a method for the treatment of a virus infection of the respiratory tract.

A further aspect of the present invention is a method for the treatment of a virus infection of the respiratory tract comprising administering to a subject in need thereof a therapeutically effective amount of a medicament comprising at least one amino acid degrading enzyme.

The medicament is suitable for use in veterinary medicine and particularly for use in human medicine.

In certain embodiments, the virus infection is a Coronavirus infection, e.g. an infection with SARS-CoV-1, SARS-CoV-2 or MERS-CoV or a Coronavirus associated with common cold, e.g. Betacoronavirus 1, for example strain OC43, strain 229E, strain NL63 or strain HKU1 or any other strain. In particular embodiments, the virus infection is an infection with SARS-CoV-2.

In further embodiments, the virus infection is an Influenzavirus infection. There are three main groups of Influenzaviruses: A, B, and C. Most common is Influenzavirus A, subdivided into serologically distinct types, e.g. H1N1, H2N2, H3N2, etc. Wild aquatic birds are hosts for many varieties of influenzavirus A. Typical seasonal flu is most commonly of the type H1N1 and H3N2. The largest flu pandemic of 1918 was caused by H1N1 type virus. Influenzavirus B is found almost exclusively in humans, while influenzavirus C infects people, dogs and pigs. While most of these viruses are also transmitted via respiratory tract, they tend to rapidly spread systemically and past the very early stages of infection may call for a systemic treatment.

The medicament is administered to a subject, particularly a human subject, suffering from a disease caused by, associated with and/or accompanied by a virus infection of the respiratory tract. In certain embodiments, the subject is suffering from a disease caused by, associated with and/or accompanied by a Coronavirus infection of the respiratory tract. In certain embodiments, the disease is SARS, Covid-19, MERS or a common cold.

All proteins are composed of 20 amino acids: aspartic acid (Asp); glutamic acid (Glu); arginine (Arg); lysine (Lys); histidine (His); asparagine (Asn); glutamine (Gln); serine (Ser); threonine (Thr); tyrosine (Tyr); alanine (Ala); glycine (Gly); valine (Val); leucine (Leu); isoleucine (Ile); proline (Pro); phenylalanine (Phe); methionine (Met); tryptophan (Trp); and cysteine (Cys). Of these twenty, nine amino acids are essential (Lys, His, Thr, Val, Leu, Ile, Phe, Met, and Trp), i.e. they cannot be synthesized by mammals. Arg is semi-essential, i.e. some synthesis is possible but not sufficient for regular requirements.

According to the present invention, the amino acid degrading enzyme is an enzyme, which is capable of degrading one or more of the above-specified amino acids. There are enzymes specific to the degradation of these amino acids; for some of them, multiple degradative pathways are known. For the purpose of killing cells infected by a virus, e.g. SARS-CoV-2, any degradative enzyme or any combination comprising at least two degradative enzymes is suitable.

In certain embodiments, the amino acid degrading enzyme is an asparagine-degrading enzyme such as asparaginase (EC 3.5.1.1). The asparaginase may be any available asparaginase, e.g. asparaginase produced in a bacterial cell such as Escherichia coli or Dickeya dadantii, including a recombinant asparaginase. In certain embodiments, the medicament may comprise asparaginase in a dissociated form, particularly in form of a monomer, e.g. as an aqueous preparation comprising urea, particularly in a concentration between about 3 mol/l to about 8 mol/l, more particularly in a concentration of about 4 mol/l to about 6 mol/l, e.g. about 5 mol/l, as described in co-owned application EP 19 178 062.6, the content of which is herein incorporated by reference.

In certain embodiments, the amino acid degrading enzyme is an arginine-degrading enzyme such as arginine deiminase (EC 3.5.3.6), arginase (EC 3.5.3.1), e.g. arginase type I or arginase type II, and arginine decarboxylase (EC 4.1.1.19), e.g. arginine decarboxylase type I or arginine decarboxylase type II. Of the two types of arginine decarboxylase—biosynthetic and biodegradative—the later has more favorable kinetic parameters to be used towards arginine depletion in humans and animals.

In certain embodiments, the amino acid degrading enzyme is a methionine-degrading enzyme such as methioninase (EC 4.4.1.11).

In certain embodiments, the amino acid degrading enzyme is a tryptophan-degrading enzyme such as tryptophan side chain oxidase (EC 1.13.99.3), e.g. tryptophan side chain oxidase type I or tryptophan side chain oxidase type II.

In certain embodiments, the medicament comprises a combination of two or more, e.g. three or four amino acid-degrading enzymes, selected from (i) an asparagine-degrading enzyme such as asparaginase (EC 3.5.1.1), (ii) an arginine-degrading enzyme such as arginine deiminase (EC 3.5.3.6), arginase (EC 3.5.3.1), e.g. arginase type I or arginase type II, and arginine decarboxylase (EC 4.1.1.19), (iii) a methionine-degrading enzyme such as methioninase (EC 4.4.1.11) and/or (iv) a tryptophan-degrading enzyme such as tryptophan side chain oxidase (EC 1.13.99.3), e.g. tryptophan side chain oxidase type I or tryptophan side chain oxidase type II.

In particular embodiments, the medicament further comprises an insulin. The insulin may be any type of insulin, e.g. human insulin, an animal insulin, an insulin analogue, including insulin analogues with short half-life and with long half-life, or an insulinotropic peptide. The preferred insulin is of short half-life, e.g. a half-life of about 12 h or less, about 8 h or less or about 4 h or less, such as Actrapid® from Novo Nordisk. The medicament may be a single composition comprising a mixture of enzyme and insulin or a combination of several compositions wherein one composition comprises an enzyme and a further composition comprises insulin.

In certain embodiments, the medicament is administered locally, e.g. orally or nasally, particularly by inhalation, whereby dilution in the bodily fluids may be avoided. For example, a single dose total volume for inhalation may be between about 1 ml and about 4 ml, preferably between about 2 ml and about 2.5 ml. Several minutes of inhalation of aerosolized enzyme(s) solution can deliver an effective dose. For example, the medicament may be administered to the upper airways such as the oral cavity, the nasal cavity, the paranasal sinus, the pharynx and/or the throat, and/or to the lower airways such as the bronchi and/or the lung.

In certain embodiments, the medicament is administered systemically, particularly in advanced stage viral infections that have spread throughout the subject's body. In such a case, the medicament can also be delivered systemically, e.g. by i.v. infusion or i.m. injection as is practiced in oncology.

The medicament may be administered to the patient in any suitable dosage form, e.g. as a rinsing solution, a spray or an aerosol, or as an injectable or infundable preparation. Typically, the medicament is administered as a pharmaceutical composition comprising the active agent and a pharmaceutically acceptable carrier or excipient. Examples of suitable carriers and excipients are well known to the skilled practitioner.

In certain embodiments, the medicament is administered in an early infection stage, e.g. in an infection stage where the upper airways such as the oral cavity, the nasal cavity, the paranasal sinus, the pharynx and/or the throat are infected, but the lower airways such as the bronchi and/or the lung are not infected. In further embodiments, the compound is administered in a late infection stage wherein the lower airways such as the bronchi and/or the lung are infected. Further, the compound may be administered in an infection stage where the subject suffers from a respiratory dysfunction and optionally is ventilated.

The medicament is administered in a therapeutically effective amount. SARS-CoV-2, like most other respiratory viruses, infects primarily, possibly exclusively, the lining cells of the respiratory tract. The total surface area of lung alveoli is about 70 m². A single cell layer covered by fluid containing lung surfactants separates the air from the capillaries on the backside of the alveoli. If the fluid layer is 1 micrometer thick, the volume in which the enzyme is to be delivered is about 10 to 100 ml. In vitro experiments have shown that amino acid degrading enzymes at 10 units/ml in the growth media have been effective in killing cancer cells but not healthy cells. Thus, in certain embodiments, particularly for local delivery, the medicament is administered in an amount comprising about 100 to about 10,000 units enzyme, particularly about 500 to about 2000 units enzyme per application.

For systemic delivery, the enzyme may be administered in a substantially higher dose. The dose to be administered to a human subject in need thereof will typically be in the range of about 250 to about 2,500 units enzyme per kg body weight per day, depending on the subject and the type and severity of the disorder to be treated. Typically, the dose to be administered to a human patient will be about 1,200 units/kg/day.

Insulin may be co-administered separately or mixed with the enzyme in a therapeutically effective amount. For local delivery, insulin may be administered in an amount of e.g. about 10 to about 500 units insulin, particularly about 50 to about 100 units insulin per application. For systemic delivery, insulin may be administered in higher amounts of e.g. about 50 to about 750 units insulin, particularly about 250 to about 500 units insulin per day. Inhalation, e.g. as dry powder, is a preferred route of delivery, but administration by i.v. infusion or i.m. injection, is also possible. When insulin is co-administered, glucose levels should be monitored, e.g. for a couple of hours after the administration and corrected if needed.

Depending on the stage and the severity of the infection, the medicament may be administered once or several times during the course of the infection. For example, the compound may be administered once a week, each second day, daily, or several times, e.g. 2 or 3-times daily.

The medicament may be administered alone or together with a further active agent. The further active agent may be selected from anti-viral agents such as remdesivir, ritonavir, and/or lopinavir, and/or interferon-beta, or antibodies against Coronavirus antigens.

According to a particularly preferred embodiment, a combination of asparaginase and an insulin, e.g. an insulin with a short half-life, is administered, e.g. as an aerosolized mixture. Since the main target in this application is only the lining cell layer in the lungs, a small volume, e.g. 0.5 to 1.0 ml of insulin, e.g. containing about 100 units/ml, mixed with about the same volume of asparaginase, e.g. containing about 2,000 units/ml, provides a base dose that can be delivered as an aerosol via inhalation during several minutes and then repeated as necessary after several hours.

1. A medicament comprising at least one amino acid degrading enzyme for use in a method for the treatment of a virus infection of the respiratory tract.

2. The medicament of claim 1 wherein the virus infection is a Coronavirus infection.

3. The medicament of claim 1 wherein the virus infection is an infection with SARS-CoV-1, SARS-CoV-2 or MERS-CoV or a Coronavirus associated with common cold, particularly an infection with SARS-CoV-2.

4. The medicament of claim 1 wherein the virus infection is an Influenzavirus infection.

5. The medicament of claim 1, which additionally comprises an insulin.

6. The medicament of claim 5, wherein the enzyme and insulin are administered as a single composition.

7. The medicament of claim 5, wherein the enzyme and insulin are administered as separate compositions.

8. The medicament of claim 1, which is administered locally, e.g. by inhalation, or systemically.

9. The medicament of claim 8, wherein a single dose volume for inhalation is between about 1 ml and about 4 ml, preferably between about 2 and about 2.5 ml.

10. The medicament of claim 1, wherein the enzyme is selected from the group consisting of (i) asparaginase, (ii) arginase, arginine deiminase or arginine decarboxylase, (iii) methioninase, and (iv) tryptophan side chain oxidase, or any combination thereof.

11. The medicament of claim 1, which comprises a dose of about 500 to about 2,000 units enzyme per application for local administration or a dose of about 250 to about 2,500 units enzyme per kg body weight per day for systemic administration.

12. The medicament of claim 5, which comprises a dose of about 50 to about 100 units insulin per application for local administration or a dose of about 250 to about 500 units insulin per day for systemic administration.

13. The medicament of claim 1, which comprises asparaginase and an insulin, particularly a mixture of asparaginase and insulin, for administration by inhalation.

14. The medicament of claim 1, which comprises asparaginase in dissociated form, particularly in a urea-containing preparation, and optionally an insulin.

15. A method for the treatment of a virus infection of the respiratory tract comprising administering to a subject in need thereof a therapeutically effective amount of a medicament comprising at least one amino acid degrading enzyme.
